(19) United States
(12) Patent Application Publication
(10) Pub. No.: US 2012/0148678 A1
(43) Pub. Date: Jun. 14, 2012

(54) SUSTAINED RELEASE OF POORLY WATER SOLUBLE ACTIVE COMPOUNDS

(75) Inventors: Deborah M. Schachter, Edison, NJ (US); Qiang Zhang, Annandale, NJ (US); Peter Van Remoortere, Princeton, NJ (US); Yufu Li, Bridgewater, NJ (US)

(73) Assignee: Advanced Technologies and Regenerative Medicine, LLC., Raynham, MA (US)

(21) Appl. No.: 12/963,012

(22) Filed: Dec. 8, 2010

(51) Int. Cl. A61K 31/5377; A61P 31/12; A61K 9/50

(52) U.S. Cl. 424/493; 514/234.5

(57) ABSTRACT

We have disclosed an implantable sustained release composition comprising, a biocompatible, biodegradable polymer, a cyclodextrin inclusion complex of a poorly water soluble pharmaceutical agent present, and a plasticizer, where the polymer is the minority phase of the formulation. Furthermore, The we disclose an implantable sustained release composition that provides a detectable plasma level of an otherwise poorly soluble drug for at least 28 days.

### FIELD OF THE INVENTION

The present invention generally relates to a sustained release formulation for poorly water soluble active compounds, which provide a long term continuous therapeutic or prophylactic treatment of disease states.

### BACKGROUND OF THE INVENTION

The sustained release of an active compound from implanted drug delivery systems has many advantages. First, implantable systems can provide a constant plasma level of the active compound for prolonged periods of time regardless of patient compliance. This is particularly important for disease states that require the delivery of anti-viral or anti-bacterial compounds. Blood circulating levels of these compounds must be maintained at constant therapeutic levels for long periods of time in order to successfully treat the infection In addition to infectious disease applications, there are other disease states or conditions that would be better served if constant plasma levels can be achieved, for example the treatment of chronic pain, psychosis, depression, and many others. Oral formulations rely on patient behavioral compliance to maintain efficacious levels of an active. Compliance can become particularly compromised by a patient's psychological state or in areas of the world with limited access to medical care. In such situations the availability of a treatment that is implanted once per month or once per two or three months can be remarkably effective both in managing each individual's disease state but also in preventing the spread of contagious disease.

Second, sustained release implantable systems can provide relatively constant plasma profiles of the therapeutic active, meaning that plasma levels of the active vary within a small range over extended periods of time and with the exception of the initial burst are devoid of the peaks and valleys that are associated with oral dosing. The high plasma levels of active achieved in the peak period of oral dosing may contribute to adverse side effects, and since oral dosage forms are ingested daily, the side effects due to the peaks and valleys are felt every day that the medications are taken. These side effects can be debilitating to the patient causing severe gastro-intestinal distress, cardiovascular toxicity, or other serious side effects that discourage compliance with treatment. However, by maintaining plasma concentrations that are significantly lower than oral formulations but still efficacious, side effects may be significantly reduced.

Third, interactions of active compounds with food and other drugs commonly used by patients can frequently occur. Plasma levels can either be significantly increased or decreased when ingesting food or other active compounds at the same time as the oral therapy. It is postulated that gastric effects are the source of the varying bioavailability of orally delivered HIV compounds, bypassing the gastro-intestinal systems can therefore avoid these interactions.

The sustained release of poorly water soluble active compounds is particularly challenging. The bioavailability of a poorly water soluble active compound from an implanted device is significantly affected by its solubility and dissolution rate since an active must first dissolve in the surrounding physiological fluid before it is bioavailable. Numerous methodologies and systems have been described to increase bioavailability of poorly soluble active compounds. One specific approach is the complexation of the active with cyclodextrin. Complexation is the association between two or more molecules to form a nonbonded entity with a well defined stoichiometry. This term is used to describe to describe the forming of an inclusion complex.

The cyclodextrin complexation of actives to enhance solubility of poorly soluble compounds for oral drug delivery is known. The melt extrusion of a physical powder blend of poorly soluble active compound, polymer, and cyclodextrin to prepare an oral dosage form with enhanced solubility of the active has been described in Fukuda, et al., Influence of Sulfobutyl Ether β-Cyclodextrin on the Dissolution Properties of Poorly Soluble Drug from Extrudates Prepared by Hot-Melt Extrusion, International Journal of Pharmaceutics 350 (2008) 188-196 and U.S. Pat. No. 6,365,188. Melt processing in both cases produced a dispersion of the active agent in the cyclodextrin. In the case of U.S. Pat. No. 6,356,5188, it was noted that this dispersion was a solid solution, and in the other case the active compound, ketoprofen, was still crystalline albeit in a different crystalline form. Solubility enhancement from dispersions of drugs in cyclodextrin can be effective, but control over the nature of the dispersion can be difficult. It is difficult to guarantee that the same morphology of active and cyclodextrin are reproduced each time, and that the ratio of active and cyclodextrin in the dispersion will always be the same. Moreover, since the active is not complexed within the cyclodextrin over the storage time there can be a change in the morphology of the dispersion potentially losing the benefit of the dispersion.

The dispersion of the active into cyclodextrin accomplished by the melt extrusion of the physical blend of powders works well for oral formulation. Oral formulations do not require the active to be actually encapsulated within the interior of the cyclodextrin cavity. A formulation containing a fine dispersion of the physical mixture of the cyclodextrin and active is enough to enhance the solubility of the poorly soluble active within the time frame required of oral ingestion. The solubility enhancement effect occurs by the increased hydration into the dosage form caused by the presence of the cyclodextrin or via a wicking effect (Bibby, et al., Mechanisms by Which Cyclodextrins Modify Drug Release from Polymeric Drug Delivery Systems, International Journal of Pharmaceutics 197, (2000) 1-11).

In contrast to oral dosage forms which are rapidly ingested, the time frame for active compounds to be eluted from implantable systems is commonly between 1 and 3 months. During this elution period, a physical mixture of the active compound and cyclodextrin can easily result in a separation of the two components with the more hydrophilic cyclodextrin eluting at a faster rate than the more hydrophobic active compound. This marked difference in elution rates of the active compound and cyclodextrin will result in a significant amount of the active agent remaining in the implant after the complete elution of the cyclodextrin. It is advantageous for implantable systems that rely on cyclodextrin to enhance the solubility of an active agent to create an inclusion complex of the cyclodextrin and the active agent to prevent this from happening. Inclusion complexes are formed by the insertion of the nonpolar molecule or the nonpolar region of one molecule (known as guest) into the cavity of another molecule or group of molecules (known as host). The major structural requirement for inclusion complexation is a snug fit of the guest into the cavity of host molecule. The cavity of host must be large enough to accommodate the guest.

For example, an inclusion complex is one complex composed of the cyclodextrin and active agent where the active agent resides within the core of the cyclodextrin molecule. The cyclodextrin molecule contains a hydrophobic core that is compatible with the hydrophobic active agent, and its outer surface is hydrophilic that will allow the inclusion complex to dissolve in aqueous solution. The inclusion complex ensures that the cyclodextrin molecule and the active agent elute together out of the implant, and in this fashion ensure that the solubility enhancement effect of the cyclodextrin is maintained during the elution period of the active from the implant.

An example of an inclusion complex is described in Yue, et al., A Novel Polymeric Chlorhexidine Delivery Device for the Treatment of Periodontal Disease, Biomaterials 25 (2004) 3743-3750. Yue et al. teaches the preparation of chlorhexidine/cyclodextrin inclusion complexes and the dispersion of the inclusion complex within a polymer matrix, particularly a hydrophobic polymer matrix to control the release rate of the active and to sustain the release of the active. Specifically, the method described here to disperse the inclusion complex into the hydrophobic polymer matrix is an emulsion-solvent evaporation technique. In this method the drug/cyclodextrin inclusion complex is sonicated with a PLGA/methylene chloride solution and the droplets harden into microparticles upon dispersion in a 1% PVA solution. Using this method, Yue et al. attempted to achieve theoretical loadings of 10, 20 and even 40%. However, the actual encapsulation efficiencies ranged from 8.1% to 16% of the theoretical values. This result means that only a fraction of the theoretical loading of the active was actually accomplished. For example, the encapsulation efficiency of the formulation that was prepared to yield a 40% (w/w) active loading was only 9.8%. Therefore the actual loading was only 4%. Yue et al. speculates that this is so due to two reasons; first, the increase in mass of the complex that must be encapsulated versus the free drug, and second, the increased osmolality of the emulsion resulted in increased leaching of the drug-complex during the hardening of the microsphere.

The dispersion of a cyclodextrin-dexamethasone inclusion complex into PLGA using a compression molding melt processing method is described in Blanco, et al., Local Release of Dexamethasone from Polymer Millirods Effectively Prevents Fibrosis after Radiofrequency Ablation, Wiley Interscience online Periodicals (2005) 174-182. Compression molding is a static method and does not have the capacity to disperse high loadings of active in a uniform manner. Homogeneity requires some dynamic mixing. The loadings described in this procedure were 1.7% active, low enough that mixing was not required, also the millirods were small enough that within the small size of the millirod there was a uniform composition. The application was the local and immediate delivery of the dexamethaxone and therefore the loading need not be large.

Bucay-Couto et al. describe the compounding of triclosan and bismuth with an effective amount of solubilizing agent into the non-degradable ethylene-vinyl acetate copolymer for the purpose of preparing ureteral stents that release triclosan in US Patent Publication Number 2007/0298069. Bucay-Couto et al. details that the triclosan and solubilizing agent are pre-mixed prior to compounding with the polymer. There appears to be no requirement that the triclosan and cyclodextrin form an inclusion complex prior to compounding with the polymer. Since the triclosan is used as an anti-microbial agent to be delivered in the local area of the stent, its efficacy is in the local space around the implant with no need for systemic circulating levels of the triclosan, and therefore the elution of high loadings of triclosan are not needed. In addition since the purpose of the triclosan is prophylactic rather than therapeutic, no systemic delivery of the drug is required rather it is necessary that the surface of the stent contain a solution of triclosan that is above the minimum inhibitory concentrations (MIC) of the particular bacteria that triclosan is active against. Consequently, there is no need for elution of high concentrations of the active compound as there would be if this were a systemic treatment for a disease.

The reason that high loadings of inclusion complexes are so challenging to disperse in a polymer is that often when preparing the inclusion complex the ratio of therapeutic agent to cyclodextrin is can be as much as 1 to 10. Therefore when preparing highly loaded melt processed dosage forms the mass to disperse in the polymer matrix is minimally 10× times the amount of therapeutic compound and if an additional excipient is required to optimize the complexation, then that translates into even more solid mass of powder that is required for dispersion into the polymer. This represents a challenge in achieving a uniform distribution of the solids throughout the polymer matrix.

Therefore, there is a need for a sustained release composition that contains high loadings of a uniformly dispersed poorly soluble agent, in the form of a cyclodextrin inclusion complex that can be prepared having an encapsulation efficiency greater than 40%. Drug encapsulation efficiency is defined as the actual drug content divided by the theoretical drug content multiplied by 100%.

### SUMMARY OF THE INVENTION

We describe herein, an implantable sustained release composition prepared from a biocompatible, biodegradable polymer, a cyclodextrin inclusion complex of a poorly water soluble pharmaceutical agent present, and a plasticizer, where the polymer is the minority phase of the formulation. The implantable sustained release composition provides a detectable plasma level of an otherwise poorly soluble drug for at least 28 days.

### BRIEF DESCRIPTION OF THE FIGURE

FIG. 1. Differential Scanning calorimetry thermogram of TM3 powder (dashed line) and the dispersion of the TM3/cyclodextrin complex in PLGA matrix prepared as described in Example 2.

### DETAILED DESCRIPTION OF THE INVENTION

We describe herein, an implantable sustained release composition comprising, a biocompatible, biodegradable polymer present in the amount from about 20% by weight to about 45% by weight of the composition; a cyclodextrin inclusion complex of a poorly water soluble pharmaceutical agent; and a plasticizer. The implantable sustained release composition provides a detectable plasma level of an otherwise poorly water soluble drug for at least 28 days.

The polymer used in the composition is one that is biocompatible and biodegradable. Biodegradable polymers readily break down into small segments when exposed to moist body tissue. The segments then either are absorbed by the body or passed by the body. More particularly, the biodegraded segments do not elicit a permanent chronic foreign body reaction, because they are absorbed by the body or passed from the body, such that no permanent trace or residual of the segment is retained by the body. Biodegradable polymers can also be referred to as bioabsorbable or bioresorbable polymers and the terms can be used interchangeably within the context of the present invention.

Suitable biocompatible, biodegradable polymers comprise aliphatic polyesters, poly(amino acids), copoly(ether-esters), polyalkylene oxalates, polyamides, poly(iminocarbonates), poly(orthoesters), polyoxaesters, polyamidoesters, polyoxaesters containing amine groups, poly(anhydrides), polyphosphazenes, and blends thereof. In one embodiment, aliphatic polyesters include, but are not limited to homopolymers and copolymers of lactide (which includes lactic acid, d-, 1- and meso lactide), glycolide (including glycolic acid), epsilon-caprolactone, p-dioxanone (1,4-dioxan-2-one), and trimethylene carbonate. In another embodiment, the biocompatible, biodegradable polymers are copolymers of lactide (which includes lactic acid, d-, 1- and meso lactide) and glycolide (including glycolic acid).

The polymer properties are important in selecting an appropriate polymer for preparing the composition. The degradation rate of these polymers is controlled by chemical structure, molecular weight of the polymer, and crystallinity. Therefore, it is necessary to evaluate these parameters when choosing the polymer to prepare the composition. The complete degradation of the polymer should take place after the dosage form has released all of the active. The preferred polymer is one that does not require processing temperatures that are at or above the thermal degradation temperature of the pharmaceutical agent. For example, the inherent viscosity of the polymer may range from about 0.1 to about 1.5 dl/g as measured at 30° C. in chloroform. In one embodiment, the inherent viscosity of the polymer may range from about 0.15 to about 1.0 dl/g. Above this range it may not possible to extrude a system with a majority phase that is solid powders and the minority phase, polymer, without increasing the temperature substantially and causing charring and decomposition of one or more of the components of the composition. One of skill in the art would be able to select the suitable polymer having appropriate properties depending upon the application, i.e. duration of sustained release and compatibility with the pharmaceutical agent for processing.

The cyclodextrin inclusion complex of a poorly water soluble pharmaceutical agent comprises a cyclodextrin, a poorly water soluble pharmaceutical agent, and a complexation aid. Poorly water soluble pharmaceutical agent is a broad description and encompasses three categories of compounds, i.e. the “very slightly soluble”, “practically insoluble” and “insoluble”. The terms “very slightly soluble”, “practically insoluble”, or “insoluble” are to be understood as defined in the following manner. A “very slightly soluble compound” requires from 1000 to 10,000 parts of solvent for 1 part of solute; a “practically insoluble” or “insoluble” compound requires more than 10,000 parts of solvent for 1 part of solute. The solvent referred to in these cases is water or aqueous buffered solutions such as, phosphate buffered saline, TRIS buffered saline, HEPES buffered saline and the like.

Pharmaceutical agent is defined to include substances that affect a biological response and that are useful to any mammal such as, humans. The term pharmaceutical agent includes, but is not limited to the following classes of drugs, therapeutic drugs, preventative drugs, and diagnostic drugs. Exemplary pharmaceutical agents for use in the composition include anti-viral drugs, narcotic pain relievers, gold salts, corticosteroids, hormones, anti-malarials, indole derivatives, drugs for the treatment of arthritis, anti-biotics, sulfur drugs, anti-tumor drugs, addiction-control drugs, weight control drugs, thyroid regulating drugs, analgesics, anti-hypertensive drugs, anti-inflammatory agents, anti-tussives, anti-epileptic, anti-depressants, antiarrhythmic agents, vasodilators, antihypertensive diuretics, anti-diabetic agents, anti-coagulants, anti-tubercular agents, agents for treating psychosis, anti-HIV drugs, anti-TB agents, agents for the treatment of hepatitis, agents for the treatment of hepatitis. The above list is not meant to be comprehensive and is merely representative of the wide variety of drugs that may be incorporated into the composition. In one embodiment, the pharmaceutical agent includes, but is not limited to drugs used in the treatment of HIV; protease inhibitors, non-nucleoside reverse transcriptase inhibitors, nucleoside reverse transcriptase inhibitors, and antiviral agents. In another embodiment, the pharmaceutical agent is an antiviral agent described in U.S. Pat. No. 7,449,463 incorporated herein by reference, such as 2-[[6-[[[2-(3-hydroxypropyl)-5-methylphenyl]amino]methyl]-2-[[3-(4-morpholinyl)propyl]amino]-1H-benzimidazol-1-yl]methyl]-6-methyl-3-pyridinol referred to herein as TM3.

The pharmaceutical agents that are suitable to be included in this composition are poorly water soluble as defined above and are those that show no appreciable decomposition at the temperatures needed to process the sustained release composition, for example at a temperature of 140° C. or less. Moreover, since the agents are complexed with the cyclodextrin in an aqueous solution they must also be stable in water. By stable, we mean that the agent does not substantially decompose, react with, or otherwise become inactive in water. In addition, the molecular structure and conformation of the active compound must be such that it can be encapsulated within the cavity of a cyclodextrin molecule. The size and shape of the molecule must be compatible with the size and shape of the cavity of the cyclodextrin molecule, and the lipophilicity of the molecule should be compatible with the lipophilicity of the interior of the cyclodextrin molecule. One of skill in the art will recognize suitable pharmaceutical agents that are stable under the processing conditions used to prepare the inclusion complex and sustained release composition.

Cyclodextrin is used to increase the solubility of the otherwise poorly water soluble pharmaceutical agent. The cylodextrin may be used to help increase the bioavailability of a poorly water soluble pharmaceutical agent by increasing the solubility of the agent in water or biological fluids, such as blood. The cyclodextrin increases the bioavailability of the poorly water soluble pharmaceutical agent only if the cyclodextrin remains in close proximity with the agent. In the case of the sustained release composition described herein, we ensure that the cyclodextrin and the agent remain in close proximity by preparing a cyclodextrin inclusion complex.

Cyclodextrins is a general term that includes alpha-, beta-, gamma-, delta-, etc. cyclodextrins and their derivatives, including cationic derivatives and derivatives with hydroxypropyl and sulfobutyl ether groups among others. Suitable cyclodextrins are those that are available commercially as a pharmaceutical grade material such as, alpha-cyclodextrin, beta-cyclodextrin, gamma-cyclodextrin, delta-cyclodextrin, hydroxypropyl-beta-cyclodextrin, sulfobutylether-beta-cyclodextrin, and the like. In one embodiment, the cyclodextrin is sulfobutylether beta-cyclodextrin.

Frequently, it is necessary to employ complexation aids when preparing a cyclodextrin inclusion complex. Suitable complexation aids include, but are not limited to polyvinylpyrrolidone (PVP), citric acid, malic acid, tartaric acid and the like. In one embodiment, the complexation aid is citric acid.

A plasticizer is used as a processing aid to facilitate the melt processing of the implantable sustained release compositions as described herein that have a higher solids content than polymer content. Suitable plasticizers are include, but are not limited to small molecules, such as dimethylsulfoxide (DMSO); poloxomers; polysorbate surfactants, phospholipid surfactants; d-alpha tocopheryl polyethylene glycol succinate (vitamin E); amphiphilic polymers; polyethyleneglycol co-polymers; poly(vinyl alcohol); and the like.

Poloxamers are nonionic triblock copolymers composed of a central hydrophobic chain of polyoxypropylene (polypropylene oxide) flanked by two hydrophilic chains of polyoxyethylene (polyethylene oxide), with varying lengths of the polymer blocks. For these copolymers are commonly named with the letter “P” (for poloxamer) followed by three digits, the first two digits give the approximate molecular mass of the polyoxypropylene core, and the last digit gives the percentage polyoxyethylene content (e.g., P407=Poloxamer with a polyoxypropylene molecular mass of 4,000 g/mol and a 70% polyoxyethylene content). Poloxamers are commercially available under the tradename PLURONIC (BASF, Mount Olive, N.J.). PLURONICs are identified by coding of the copolymers. The coding starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the polyoxypropylene hydrophobe. The last digit, when multiplied by 10, indicates the approximate ethylene oxide content in the molecule (e.g., F127=PLURONIC with a polyoxypropylene molecular weight of 3,600 g/mol and a 70% polyoxyethylene content). PLURONIC F127 corresponds to poloxamer P407 (P407).

In one embodiment, the poloxamers have a polyoxypropylene molecular weight that is in the range of about 3,000 to about 4,800 g/mol and a polyoxyethylene content that is in the range of about 70% to about 80%. In one embodiment, the plasticizer is DMSO.

Polysorbate surfactants are polyoxyethylene derivatives of sorbitan esterified with fatty acids and are commercially available under the tradename TWEEN (Uniqema Americas LLC, Wilmington, Del.) and are distinguished from the other members polysorbate family by the length of the polyoxyethylene chain and the fatty acid ester moiety. In one embodiment, the polysorbate surfactant includes, but is not limited to polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), and the like.

The implantable sustained release composition is prepared by making the cyclodextrin inclusion complex of the poorly water soluble pharmaceutical agent and then compounding the cyclodextrin inclusion complex into the biocompatible, biodegradable polymer.

The cyclodextrin inclusion complex is prepared by dissolving the cyclodextrin and the poorly soluble pharmaceutical agent in water or aqueous buffered solution (as described above), optionally with complexation aid(s), and mixing until dissolved at about room temperature. The temperature can be elevated to accelerate the rate of complexation, but should not exceed the thermal stability temperature of the pharmaceutical agent. The correct ratio of each component is necessary to achieve the encapsulation of the active into the cyclodextrin and can be experimentally determined by one of the skill in the art. The cyclodextrin inclusion complex is then isolated by drying thereby removing the water. The water may be removed by conventional drying methods, such as rotoevaporation, vacuum drying, lyophilization and the like. The drying method that is selected should take into consideration the stability of the pharmaceutical agent under the drying conditions.

The cyclodextrin inclusion complex is then melt blended into a biocompatible, biodegradable polymer using conventional polymer processing methods. For example, the polymer is placed in a conventional twin-screw mixer, that is pre-heated to a temperature that is above the glass transition temperature, for example at a minimum of 10° C. above the Tg of the polymer. In one embodiment, the mixer is preheated to a temperature of about 50° C. to about 150° C. The temperature is selected to minimize degradation of any components. The screws are pre-set to a speed that provides efficient mixing, for example from about 50 rpm to about 100 rpm. A plasticizer such as dimethylsulfoxide is added to the composition within the range of about 3% (w/w) to about 10% (w/w) of the weight of the composition to ensure processibility of the polymer system with such high solids content. The plasticizer is combined with the dried cyclodextrin inclusion complex and the mixture is then added to the pre-heated polymer. Mixing continues at the set temperature until a uniform mixture is achieved. The mixture is then removed from the twin-screw mixer, cooled to room temperature and fed into a compounder, again set at a temperature and mixing speed that is effective to extrude the composition. Higher temperature and greater speed, for example temperatures of from about 100° C. to about 150° C. and mixing speeds of from about 80 rpms to about 100 rpm, are used for this step in order to force the material through the die at the exit orifice. The die can be in any suitable shape for extruding fiber strands such as, triangular, square, round, oval, or rectangular shaped and the like. The die may also be flat, i.e. in the shape of a slit to provide a ribbon shaped fiber or strand. In our studies a round die was used to provide cylinders that can easily be injected into the subcutaneous space. The diameter of the die is one that provides sustained release compositions of suitable size for injection and placement with a conventional trocar or needle. For example, the die used here was 2 mm, thus the specimens were cylinders having approximately a 2 mm diameter. One of skill in the art of polymer processing can identify suitable conditions for blending and extruding the composition such that the pharmaceutical agent substantially maintains its activity and that the cyclodextrin inclusion complex is distributed throughout the composition.

The poorly water soluble pharmaceutical agent is present in the controlled release composition in a range of from about 1% by weight to about 40% by weight. In one embodiment, the pharmaceutical agent is present in the composition in a range of from about 2% by weight to about 20% by weight. The polymer is present in the sustained release composition in a range of from about 20% by weight to about 45% by weight. Therefore, the polymer is present in an amount that is less than the total sum of the weight percents of solids, i.e. of the pharmaceutical agent, cyclodextrin, and complexation aids.

In addition to the large amount of solids that we are able to uniformly distribute in the polymer during extrusion, the process results in a very high encapsulation efficiency of the poorly water soluble pharmaceutical agent. The encapsulation efficiency is measured in the following manner. A preweighed amount of the formulation is dissolved in a common solvent that dissolves the polymer and the poorly water soluble pharmaceutical agent. An aliquot of the solution is then analyzed by a high performance liquid chromatography (HPLC) method specifically developed for the poorly water soluble pharmaceutical agent. The actual poorly water soluble pharmaceutical agent content that results from the HPLC assay is divided by the theoretical poorly water soluble pharmaceutical agent content and multiplied by 100 percent to obtain the encapsulation efficiency. We were able to achieve encapsulation efficiencies ranging from about 85% to about 110%.

The advantages of the invention lie in the fact that a poorly soluble pharmaceutical agent can be made systemically bioavailable for extended periods of time from an implanted sustained release formulation. Implantable formulations that are dosed once a month or perhaps even more infrequently require large amounts of active in each dose to last for the entire dosing period. A poorly soluble pharmaceutical agent that requires complexation increases the formulation challenge since the large amount of drug powder plus the added materials to promote the complexation need to be distributed in the biocompatible, biodegradable polymer. Moreover, the complex composed of drug and cyclodextrin must maintain its stability for the duration of therapy in order to maintain the bioavailability of the pharmaceutical agent. The compositions reported here and processed according to the prescribed process method yield sustained release compositions with a uniform distribution of solids, yet can accommodate the large mass of powders (pharmaceutical agent plus complexation materials) and still provide bioavailable plasma levels of the poorly water soluble pharmaceutical agent. The method also ensures that the encapsulation efficiency is high ensuring that the sustained release composition can reproducibly deliver the most pharmaceutical agent possible within the volume of the formulation.

### EXAMPLES

### Example 1

The anti-viral agent, 2-[[6-[[[2-(3-hydroxypropyl)-5-methylphenyl]amino]methyl]-2-[[3-(4-morpholinyl)propyl]amino]-1H-benzimidazol-1-yl]methyl]-6-methyl-3-pyridinol, referred to as TM3, was tested for solubility in water as a free powder, in a cylodextrin inclusion complex and as a sustained release formulation.

Two respective samples of 3.9 mg of TM3 (Janssen Pharmaceutical, Beerse, BE) were weighed out and placed into 20 ml of distilled water. The samples were placed in a water shaker bath heated to 37° C. and shaken overnight. The next morning samples were filtered through a 0.25 micron filter and injected into an HPLC to determine the TM3 concentration in water which were 0.00004 mg/ml and 0.00011 mg/ml, respectively. These results indicate the insoluble nature of the compound. The procedure described in Example 2 was used to prepare the cyclodextrin inclusion complex of TM3 in duplicate and the solubility of the respective samples was determined. The resulting solubilities were 0.120 mg/ml and 0.115 mg/ml, respectively. To observe whether the enhanced solubility effect was maintained even after the complex was dispersed in a polymer, the complex was dispersed into PLGA using the melt processing method described in Example 2. Two samples of the extrudate were tested for TM3 solubility in the manner described above. After overnight incubation in 20 ml of water at 37° C. the polymer formulation containing the complex exhibited a TM3 concentration of 0.036 and 0.021 mg/ml in the elution media. In contrast, the polymer formulation containing the dispersion of the TM3 powder exhibited a TM3 concentration of 0.00004 mg/ml for both samples in the elution media.

The similarity in concentrations of TM3 exhibited by the polymer dispersion of the TM3 powder and the TM3 powder itself, indicates that merely dispersing in the polymer with added DMSO does not lead to solubility enhancement and the diffusion of TM3 from the polymer is significantly limited by its low aqueous solubility.

### Example 2

The cyclodextrin inclusion complex of TM3 was prepared using the following procedure. Five grams of TM3 (Janssen Pharmaceutical, Beerse, BE) was weighed with 3.76 grams of citric acid (Sigma-Aldrich, St Louis, Mo.) and 25.00 grams of cyclodextrin sold under the tradename CAPTISOL (CyDex Pharmaceuticals, Inc., Lenexa, Kans.) and placed in a 1000 ml round bottom flask with 100 ml of distilled water. A magnetic stir-bar was added and the solution was stirred for 15 minutes at room temperature until clear and amber. The solution was dried on a rotary evaporator at 95° C. for 30 minutes under aspirator vacuum. The product was scraped from the flask and dried at 95° C. in a vacuum oven for 2 hours, and ground with mortar and pestle to a fine powder. The powder was vacuum dried for an additional 2 hours at 95° C. followed by vacuum drying overnight at room temperature. A sample of the inclusion complex was tested by HPLC for TM3 content, and the results indicated a 15% (w/w) concentration of TM3 in the complex. The sustained release composition was then prepared by feeding 9 grams of poly(lactide-co-glycolide) (50/50 mole ratio) (Surmodics Pharmaceuticals, Birmingham, Ala., LP-207, IV=0.18 dl/g) into a 30 cc twin screw mixer (Brabender, GMBH & Co. Duisburg, Germany) that was pre-set to 100° C. and 60 rpm. Eighteen grams of the cyclodextrin inclusion complex of TM3 which had been pre-mixed with 3 grams of DMSO (methyl sulfoxide anhydrous ACROS, Morris Plains, N.J.) were added to the molten polymer. Mixing continued for an additional 5 minutes. The mixture was then removed from the mixer, cooled to room temperature and fed into a compounder, (Daca Instrument, Santa Barbara, Calif.) that was pre-set to 100° C. and running at 120 rpm. Extrudate emerged from the die as strands of diameter 1.5-2 mm. Samples were cut into 2 mm lengths and stored at room temperature in a nitrogen atmosphere.

### Example 3

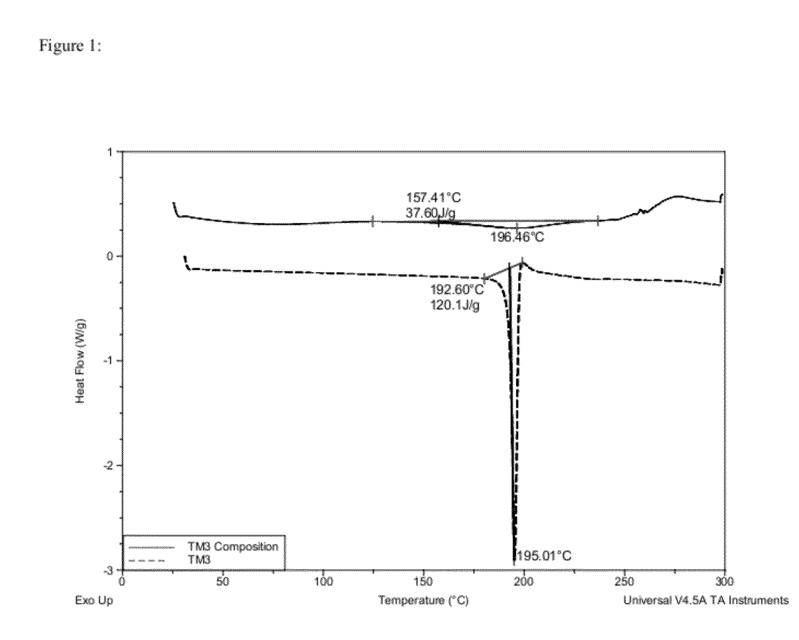

Thermal analysis was performed on the neat TM3 powder and the sustained release composition prepared as just described in Example 2. Samples were placed in respective DSC pans, crimped and heated in a nitrogen environment at a rate of 5° C./minute from room temperature to 300° C. The thermograms are shown in FIG. 1. The TM3 powder has a sharp melting point at 195° C., with a heat of melting of 120 J/g. The thermogram of the sustained release composition does not exhibit a clear melting point at all for TM3.

Considering that the loading of TM3 in the sustained release composition is 10%, melting transition would be apparent if the active compound were present in the sustained release composition as a crystalline material. The lack of a clear melting transition indicates the amorphous nature of the cyclodextrin inclusion complex of TM3 dispersed in the polymer.

### Example 4

A cyclodextrin inclusion complex of TM3 (Janssen Pharmaceutical, Beerse, BE) was prepared as described in Example 2 using 5 grams of TM3, 2.10 grams of citric acid (Sigma-Aldrich, St Louis, Mo.), and 12.5 grams of cyclodextrin (sold under the tradename CAPTISOL (CyDex Pharmaceuticals, Inc, Lenexa, Kans.). The dried complex was used to prepare a sustained release composition.

Ten grams of PLGA 75/25 (IV=0.21) (Surmodics Pharmaceuticals, Birmingham, Ala.) were fed into a twin screw mixer (Brabender GMBH & Co., Duisburg, Germany) that was pre-set to 100° C. and 60 rpm. Nineteen grams of the complex were pre-mixed with 1 gram of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set at 120° C. and 100 rpm. Extrudate emerged from the die as strands 2 mm in diameter. Mixture was successfully processed, TM3 content analysis of this lot was completed using HPLC and the concentration of TM3 in the sustained release composition was 16% (w/w). The encapsulation efficiency of this formulation was 100%. Samples were cut into 2 mm lengths and stored under nitrogen at ambient temperature.

### Example 5

A cyclodextrin inclusion complex of TM3 was prepared as described in Example 2 using 5 grams of TM3 (Janssen Pharmaceutica, Beerse, BE), 3.76 grams of citric acid (Sigma-Aldrich, St Louis, Mo.), and 12.5 grams of cyclodextrin (sold under the tradename CAPTISOL, CyDex Pharmaceuticals, Inc, Lenexa, Kans.). The dried complex was used to prepare a sustained release composition.

Ten grams of PLGA 75/25 (IV=0.21) (Surmodics Pharmaceuticals, Birmingham, Ala.) were fed into a twin screw mixer (Brabender GMBH & co., Duisburg, Germany) that was pre-set to 100° C. and 60 rpm. Nineteen grams of the complex were pre-mixed with 3 grams of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set to 120° C. and 100 rpm. Extrudate emerged from the die as strands 2 mm in diameter. Mixture was successfully processed, the TM3 content analysis of this lot was completed using HPLC and the concentration of TM3 in the sustained release composition was 16% (w/w). The encapsulation efficiency of this formulation is 110%. Samples were cut into 2 mm lengths and stored under nitrogen at ambient temperature.

### Example 6

A cyclodextrin inclusion complex of TM3 was prepared as described in Example 2 using 5 grams of TM3 (Janssen Pharmaceutica, Beerse, BE), 2.10 grams of citric acid (Sigma-Aldrich, St Louis, Mo.), 25.0 grams of cyclodextrin (sold under the tradename CAPTISOL (CyDex Pharmaceuticals, Inc, Lenexa, Kans.). The dried complex was used to prepare a sustained release composition.

Ten grams of PLGA 75/25 (IV=0.70) (Suromdics Pharmaceuticals, Birmingham, Ala.) were fed into in a twin screw mixer (Brabender GMBH & co., Duisburg, Germany)) that was pre-set to 100° C. and 60 rpm. Nineteen grams of the complex was pre-mixed with 1 gram of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.)) that was pre-set at 120° C. and 100 rpm. Although barrel temperature was increased from 120 to 140° C. it was not possible to process this mixture successfully.

Similarly to previous examples, the solids content, 63% (w/w), was greater than the polymer fraction. The polymer used in this example is the polymer with the higher inherent viscosity, and therefore more difficult to extrude than the lower inherent viscosity polymer used in some of the other formulations. At the same time, in this formulation the plasticizer is reduced to the minimum fraction of 3% (w/w) reducing its ability to plasticize the formulation and thereby facilitate extrusion. This would be similar to the formulation in Example 8 but in this case the mole ratio of PLA to PGA is 75/25 instead of 50/50 as in Example 8. The polymer with higher PLA content has a higher glass transition temperature, with a range of 48-53° C. versus the PLGA 50/50 with 43-48° C. This example illustrates that at the point where the lowest limit of plasticizer is used, and an inherent viscosity that is closer to the upper limit in such a situation where the solids content is larger than the polymer content, small differences in glass transition temperature can render the entire composition challenging to process.

### Example 7

A cyclodextrin inclusion complex of TM3 was prepared as described in Example 2 using 5 grams of TM3 (Janssen Pharmaceutica, Beerse, BE), 3.76 grams of citric acid (Sigma-Aldrich, St Louis, Mo.), 25.0 grams of cyclodextrin (sold under the tradename CAPTISOL, CyDex Pharmaceuticals, Inc, Lenexa, Kans.). The dried complex was used to prepare a sustained release composition.

Ten grams of PLGA 75/25 (IV=0.70) (Surmodics Pharmaceuticals, Birminham, Ala.) were fed into in a twin screw mixer (Brabender GMBH & Co. Duisberg, Germany) that was pre-set to 100° C. and 60 rpm. Nineteen grams of the complex was pre-mixed with 3 grams of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set at 140° C. and 100 rpm. Extrudate emerged from the die as strands 2 mm in diameter. Mixture was successfully processed. The content analysis of this lot was completed using HPLC and the concentration of TM3 in the sustained release composition was 11.5% (w/w). The encapsulation efficiency was 107%. Samples were cut into 2 mm lengths and stored under nitrogen at ambient temperature.

### Example 8

A cyclodextrin inclusion complex of TM3 was prepared as described in Example 2 using 5 grams of TM3 (Janssen Pharmaceutica, Beerse, BE), 3.76 grams of citric acid (Sigma-Aldrich, St Louis, Mo.), 12.5 grams of cyclodextrin (sold under the tradename CAPTISOL, CyDex Pharmaceuticals, Inc, Lenexa, Kans.). The dried complex was used to prepare a sustained release composition.

Ten grams of PLGA 50/50 (IV=0.79) (Surmodics Pharmaceuticals, Birmingham, Ala.) were fed into in a twin screw mixer (Brabender GMBH & Co. Duisberg, Germany) that was pre-set to 100° C. and 60 rpm. Nineteen grams of the complex was pre-mixed with one gram of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set at 120° C. and 100 rpm. Extrudate emerged from the die as strands 2 mm in diameter. The mixture was successfully processed, content analysis of this lot was completed using HPLC and the concentration of TM3 in the sustained release composition was 13% (w/w). The encapsulation efficiency was 86%. Samples were cut into 2 mm lengths and stored at room temperature in a nitrogen atmosphere.

### Example 9

A cyclodextrin inclusion complex of TM3 was prepared as described in Example 2 using 5 grams of TM3 (Janssen Pharmaceutica, Beerse, BE, 2.10 grams of citric acid (Sigma-Aldrich, St Louis, Mo.), 12.5 grams of cyclodextrin (sold under the tradename CAPTISOL, CyDex Pharmaceuticals, Inc, Lenexa, Kans.). The dried complex was used to prepare a sustained release composition.

Ten grams of PLGA 50/50 (IV=0.79) (Surmodics Pharmaceuticals, Birmingham, Ala., LP-220) were fed into in a twin screw mixer (Brabender GMBH & Co. Duisberg, Germany) that was pre-set to 100° C. and 60 rpm. Nineteen grams of the complex were pre-mixed with 3 grams of DMSO and added to the heated polymer at the set conditions. Mixing continued for 5 minutes. Subsequently, the mixture was removed from the mixer and placed into a compounder (Daca Instruments, Santa Barbara, Calif.) that was pre-set at 120° C. and 100 rpm. Extrudate emerged from the die as strands 2 mm in diameter. The mixture was successfully processed, the content analysis of this lot was completed using HPLC and the concentration of TM3 in the sustained release composition was 15%. The encapsulation efficiency was 98%. Samples were cut into 2 mm lengths and stored at room temperature in a nitrogen atmosphere.

### Example 10

A cyclodextrin inclusion complex of TM3 was prepared as described in Example 2 using 5 grams of TM3 (Janssen Pharmaceutica, Beerse, BE), 3.76 grams of citric acid (Sigma-Aldrich, St Louis, Mo.), 25.0 grams of cyclodextrin (sold under the tradename CAPTISOL, CyDex Pharmaceuticals, Inc, Lenexa, Kans.). The dried complex was used to prepare a sustained release composition.

Ten grams of PLGA 50/50 (IV=0.18) (Surmodics Pharmaceuticals, Birmingham, Ala.) were fed into twin screw mixer (Brabender GMBH & Co. Duisberg, Germany)) that was pre-set to 100° C. and 60 rpm. Nineteen grams of the complex were pre-mixed with one gram of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set at 140° C. and 100 rpm. Extrudate emerged from the die as strands 2 mm in diameter. The mixture was successfully processed, content analysis of this lot was completed using HPLC and the concentration of TM3 in the sustained release composition was 12% (w/w). The encapsulation efficiency was 108%. Samples were cut into 2 mm lengths and stored at room temperature in a nitrogen atmosphere.

### Example 11

A cyclodextrin inclusion complex of TM3 was prepared as described in Example 2 using 5 grams of TM3 (Janssen Pharmaceutica, Beerse, BE), 2.10 grams of citric acid (Sigma-Aldrich, St Louis, Mo.), 25 grams of cyclodextrin (sold under the tradename CAPTISOL, CyDex Pharmaceuticals, Inc, Lenexa, Kans.). The dried complex was used to prepare a sustained release composition.

Ten grams of PLGA 50/50 (IV=0.18) (Surmodics Pharmaceuticals, Birmingham, Ala.) were fed into in twin screw mixer (Brabender GMBH & Co. Duisberg, Germany) that was pre-set to 100° C. and 60 rpm. Nineteen grams of the complex were pre-mixed with three grams of DMSO (methyl sulfoxide anhydrous, ACROS, Morris Plains, N.J.) and added to the heated polymer at the set conditions. Mixing continued for 5 minutes. Subsequently, the mixture was removed from the mixer and placed into a compounder (Daca Instruments, Santa Barbara, Calif.) that was pre-set at 140° C. and 100 rpm. Extrudate emerged from the die as strands 2 mm in diameter. The mixture was successfully processed, content analysis of this lot was completed using HPLC and the concentration of TM3 in the sustained release composition was 12% (w/w). The encapsulation efficiency was 107%. Samples were cut into 2 mm lengths and stored at room temperature in a nitrogen atmosphere.

### Example 12

The sustained release capability of the formulation was tested in vivo in a rat model. A cyclodextrin inclusion complex of TM3 was prepared by adding 6.00 grams of TM3 (Janssen Pharmaceutica, Beerse, BE), to a 1000 ml round bottom flask with 4.51 grams of citric acid (Sigma-Aldrich, St Louis, Mo.), 30.00 grams of cyclodextrin (sold under the trandename CAPTISOL, CyDex, Inc., Lenexa, Kans.) and 120 ml of distilled water. A magnetic stir-bar was added and the solution was stirred for 15 minutes at room temperature until clear and amber. The solution was dried on the rotary evaporator at 95° C. using aspirator vacuum for 2 hours, ground with a mortar and pestle to a fine powder and subsequently dried in a vacuum oven for 2 hours at 95° C. then continued drying overnight at room temperature. The resulting mass of the cyclodextrin inclusion complex was 38.83 grams. Eighteen grams of the cyclodextrin inclusion complex of TM3 was pre-mixed with 3 grams of DMSO (methyl sulfoxide anhydrous, ACROS, Morris Plains, N.J.). Nine grams of PLGA 50/50 (Surmodics Pharmaceuticals, Birmingham, Ala., LP-207, IV=0.18 dl/g) were fed into a twin screw mixer (Brabender GMBH & Co. Duisburg, Germany) mixing bowl that was pre-set to 100° C. and 60 rpm. The inclusion complex/DMSO mixture was added to the heated polymer and mixing continued at the set conditions for an additional 5 minutes. The mixture was removed from the mixing bowl, cooled in ambient conditions, and fed into a compounder (Daca Instruments, Santa Barbara, Calif.) (pre-set to 120° C., 100 rpm). The mixture was extruded through a die and collected in the form of strands having an approximately 2 mm diameter. The extrudates were pelletized into pellets having weights of between 60-65 mg. The TM3 content in the pellets was determined by HPLC and found to be 13% (w/w) or about 7-8 mg. Pellets were implanted into male rats using a subcutaneous injection by trocar into the scapular region. The TM3 dose was 20-23 mg/kg. Blood samples were taken at pre-determined time points and the TM3 was extracted from the samples. The blood plasma levels of TM3 are summarized in Table 1.

A first control formulation composed of TM3 alone and PLGA was prepared in the following manner. Twelve grams of PLGA 50/50 (Surmodics Pharmaceuticals, Birmingham, Ala., LP-207, IV=0.18 dl/g) was placed in a twin screw mixer (Brabender GMBH & Co., Duisburg, Germany) mixing bowl that was pre-heated to 100° C. and 60 rpm. Eighteen grams of TM3 (Janssen Pharmaceutica, Beerse, BE) were added to the heated polymer. Mixing continued for an additional 5 minutes. The mixture was removed from the mixing bowl and fed into a compounder (Daca Instruments, Santa Barbara, Calif.) that was pre-set to 100° C. and 100 rpm. The formulation was extruded through a 2 mm round die and collected as strands having a diameter of approximately 2.0 mm. The formulation was pelletized into pellets that weighed 12-13 mg. HPLC analysis for TM3 content confirmed that the TM3 content was 60% (w/w). The content of TM3 in each pellet ranged from 7.3-7.7 mg. Pellets were implanted using subcutaneous injection by using a trocar into the scapular region of the rats. The weight of the rats was approximately 350 grams providing a dose of 20-23 mg/kg. Blood samples were taken at pre-determined time points and the TM3 was extracted from the samples. The blood plasma levels of TM3 are summarized in Table 1.

A second control formulation composed of TM3, PLGA, and DMSO was prepared in the following manner. Nine grams of PLGA (Surmodics Pharmaceuticals, Birmingham, Ala., LP-207, IV=0.18 dl/g) were placed into a twin screw mixer (Brabender GMBH & Co., Duisburg, Germany) mixing bowl that was pre-set to 100° C. and 60 rpm. Eighteen grams of TM3 (Janssen Pharmaceutica, Beerse, BE) were pre-mixed with 3 grams of DMSO (methyl sulfoxide anhydrous, ACROS, Morris Plains, N.J.) and added to the heated polymer. Mixing continued at for an additional 5 minutes. Subsequently, the mixture was removed from the mixing bowl, cooled to room temperature and fed to a compounder (Daca Instruments, Santa Barbara, Calif.) that was pre-set to 100° C. and 100 rpm. The formulation was extruded through a 2 mm die and collected as strands of having a diameter of approximately 2 mm. The formulation was pelletized into pellets that weighed 12-13 mg. Content analysis for TM3 was assayed by HPLC and found to be 60% (w/w), the content of TM3 in each test pellet ranged from 7.6-7.9 mg. Pellets were implanted into the scapular region of the rats using the trocar method. The rats weighed approximately 350 grams providing a dose of 20-23 mg/kg. Blood samples were taken at pre-determined time points and the TM3 was extracted from the samples. The blood plasma levels of TM3 are summarized in Table 1.

TABLE 1
Plasma Concentration of TM3 (ng/ml)

| Animal | Implant | 1 | 3 | 8 | 24 | 48 | 96 | 7 | 13 | 28 |
|---|---|---|---|---|---|---|---|---|---|---|
| number | composition | hour | hour | hour | hour | hour | hour | day | day | day |
| 6M | TM3/ | 185 | 105 | 22.4 | 3.77 | 1.75 | 0.491 | 1.05 | 0.337 | 0.630 |
| 7M | Cyclodextrin/ | 165 | 159 | 32.3 | 4.77 | 2.20 | 1.23 | 2.88 | 2.64 | BQL |
| 8M | citric acid/ | 344 | 144 | 44.0 | 5.34 | 2.08 | 0.718 | 1.53 | 0.636 | 0.351 |
| 9M | DMSO/PLGA | 95.5 | 170 | 24.2 | 4.79 | 1.63 | 0.975 | 3.09 | 1.84 | 0.647 |
| 10M | | 332 | 264 | 91.3 | 13.1 | 5.34 | 1.42 | 1.35 | BQL <0.2 | 0.554 |
| 06M | TM3/PLGA | BQL <2.0 | BQL <2.0 | BQL <2.0 | BQL <2.0 | BQL <2.0 | BQL <2.0 | BQL <0.2 | BQL <2.0 | BQL <0.2 |
| 07M | | BQL <2.0 | BQL <2.0 | BQL <2.0 | BQL <2.0 | BQL <2.0 | BQL <2.0 | BQL <0.2 | BQL <2.0 | BQL <0.2 |
| 08M | | BQL <2.0 | BQL <2.0 | BQL <2.0 | BQL <2.0 | BQL <2.0 | BQL <2.0 | BQL <0.2 | BQL <2.0 | BQL <0.2 |
| 09M | | BQL <2.0 | BQL <2.0 | BQL <2.0 | BQL <2.0 | BQL <2.0 | BQL <2.0 | BQL <0.2 | BQL <2.0 | BQL <0.2 |
| 10M | | BQL <2.0 | BQL <2.0 | BQL <2.0 | BQL <2.0 | BQL <2.0 | BQL <2.0 | BQL <0.2 | BQL <2.0 | BQL <0.2 |
| 6M | TM3/DMSO/ | BQL <2.0 | BQL <2.0 | BQL <2.0 | BQL <2.0 | BQL <2.0 | BQL <2.0 | 0.436 | BQL <2.0 | BQL <0.2 |
| 7M | PLGA | BQL <2.0 | BQL <2.0 | BQL <2.0 | BQL <2.0 | BQL <2.0 | BQL <2.0 | BQL <0.2 | BQL <2.0 | BQL <0.2 |
| 8M | | BQL <2.0 | BQL <2.0 | BQL <2.0 | BQL <2.0 | BQL <2.0 | BQL <2.0 | 17.7 | BQL <2.0 | BQL <0.4 |
| 9M | | BQL <2.0 | BQL <2.0 | BQL <2.0 | BQL <2.0 | BQL <2.0 | BQL <2.0 | 0.415 | BQL <2.0 | BQL <0.2 |
| 10M | | BQL <2.0 | BQL <2.0 | BQL <2.0 | BQL <2.0 | BQL <2.0 | BQL <2.0 | 0.394 | BQL <2.0 | BQL <0.2 |

BQL = below quantitative limit

The sustained release formulation of the invention showed a large burst within the first day, however, measurable plasma levels of TM3 were observed over the 28 days that samples were taken. In comparison, the first control sample did not show any detectable levels of TM3 at any of the time points. At seven days and at 28 days the detection limit for samples was reduced to 0.2 ng/ml but even at this low level no TM3 was observed. The second control sample did not show any detectable levels of TM3 until the 7 day time point for four out of the five test animals. Although the limit of quantitation was reduced to 0.2 ng/ml for the 28 day time point, no TM3 was detected after the 7 day time point. Therefore, we have shown that the sustained release formulation of the invention provides a sustained release of TM3 as well as bioavailability over the course of at least 28 days.

### Example 13

The patient is prepared by laying on his/her back with the arm least used (e.g. left arm for a right-handed person) flexed so the physician has a ready access to the inner aspect of the upper arm. The patient's arm is propped with pillows so the patient can easily hold that position. The implant is loaded into the cannula of the insertion tool (trocar with cannulae) prior to prepping the insertion field and insertion site. The insertion area is first swabbed with povidone-iodine swabs, a fenestrated drape is placed over the insertion site. The method of anesthesia utilized should be determined at the discretion of the physician. A topical lidocaine cream can be used to anesthetize the area prior to injection of local anesthetic. The local anesthetic is injected starting at the planned incision site, followed by infiltration up to the length of the implant (25-30 mm). A 2-3 mm incision is made subcutaneously with a scalpel. The trocar is used to insert the implant into the prepared site. Insertion site can be closed using one or two sutures.

We claim:

1. An implantable sustained release composition comprising, a biocompatible, biodegradable polymer present in the amount of from about 20% by weight to about 45% by weight of the composition; and a cyclodextrin inclusion complex of a poorly water soluble pharmaceutical agent; and a plasticizer; wherein the sustained release composition has an encapsulation efficiency of the poorly water soluble pharmaceutical agent in the range of from about 85% by weight to about 100% by weight.

2. The composition of claim 1, where the biocompatible, biodegradable polymer is an aliphatic polyester selected from the group consisting of homopolymers and copolymers of lactide, glycolide, epsilon-caprolactone, p-dioxanone, and trimethylene carbonate.

3. The composition of claim 2 where the biocompatible, biodegradable polymer is selected from the group consisting of copolymers of lactide and glycolide.

4. The composition of claim 2 where the biocompatible, biodegradable polymer has an inherent viscosity in chloroform at 30° C. of from about 0.5 dL/g to about 1.75 dL/g.

5. The composition of claim 1, where the cyclodextrin inclusion complex of a poorly water soluble pharmaceutical agent comprises a cyclodextrin, a poorly water soluble pharmaceutical agent, and a complexation aid.

6. The composition of claim 5, where the cyclodextrin is selected from the group consisting of alpha-cyclodextrin, beta-cyclodextrin, gamma-cyclodextrin, delta-cyclodextrin, hydroxypropyl-beta-cyclodextrin, and sulfobutylether-beta-cyclodextrin.

7. The composition of claim 6, where the cyclodextrin is sulfobutylether beta-cyclodextrin.

8. The composition of claim 5, where the poorly water soluble pharmaceutical agent is selected from the group consisting of narcotic pain relievers, gold salts, corticosteroids, hormones, anti-malarials, indole derivatives, drugs for the treatment of arthritis, anti-biotics, sulfur drugs, anti-tumor drugs, addiction-control drugs, weight control drugs, thyroid regulating drugs, analgesics, anti-hypertensive drugs, anti-inflammatory agents, anti-tussives, anti-eleptics, anti-depressants, antiarrhythmic agents, vasodilators, antihypertensive diuretics, anti-diabetic agents, anti-coagulants, anti-tubercular agents, agents for treating psychosis, anti-HIV drugs, anti-TB agents, agents for the treatment of hepatitis, and agents for the treatment of hepatitis.

9. The composition of claim 8 where the poorly water soluble pharmaceutical agent is an anti-viral.

10. The composition of claim 9 where the anti-viral is 2-[[6-[[[2-(3-hydroxypropyl)-5-methylphenyl]amino]methyl]-2-[[3-(4-morpholinyl)propyl]amino]-1H-benzimidazol-1-yl]methyl]-6-methyl-3-pyridinol.

11. The composition of claim 5 where the complexation aid is selected from the group consisting of polyvinylpyrrolidone, citric acid, malic acid, tartaric acid.

12. The composition of claim 11 where the complexation aid is citric acid.

13. The composition of claim 1 where the plasticizer is selected from the group consisting of dimethylsulfoxide, poloxomers, polysorbate surfactants, phospholipid surfactants, d-alpha tocopheryl polyethylene glycol succinate, amphiphilic polymers, polyethylene glycol co-polymers, and poly(vinyl alcohol).

14. The composition of claim 13 where the plasticizer is dimethylsulfoxide.
